(19) United States
(12) Patent Application Publication
(10) Pub. No.: US 2006/0246080 A1
(43) Pub. Date: Nov. 2, 2006

(54) Multipathogen and monopathogen protection against the bacterial and viral infections associated with biological warfare, cardiac diseases, and cancers

(76) Inventors: Ken Alibek, Fairfax, VA (US); Brian Pickering, Manassas Park, VA (US); Vyvy Pham, Bethesda, MD (US); Svetlana Hopkins, Arlington, VA (US); Aiguo Wu, Arlington, VA (US)

Correspondence Address: HOGAN & HARTSON L.L.P., 500 S. GRAND AVENUE, SUITE 1900, LOS ANGELES, CA, 90071-2611, US

(21) Appl. No.: 11/041,881

(22) Filed: Jan. 25, 2005

Related U.S. Application Data
(60) Provisional application No. 60/612,616, filed on Sep. 24, 2004.
(60) Provisional application No. 60/615,591, filed on Oct. 5, 2004.
(60) Provisional application No. 60/622,112, filed on Oct. 27, 2004.

(51) Int. Cl. G01N 33/53; G06F 19/00; A61K 39/00

(52) U.S. Cl. 424/184.1; 435/7.1; 702/19

(57) ABSTRACT

A method for developing vaccines and treatments against pathogens associated with biological warfare, cardiac disease, cancer, and emerging infectious diseases is to analyze the sequences of proteases of the pathogens and determine which peptide antigens will be useful in treating or preventing the effects of one or more pathogens as vaccines or to provide passive immunity. The sequences of the selected proteases are used to screen the genomes of other pathogens to produce broad spectrum vaccines and treatments that target one or several pathogens. Amino acid sequences of the peptide antigens and immunogenic compositions comprising them are also contemplated.

### REFERENCE TO CROSS-RELATED APPLICATIONS

This application claims the benefit of U.S. provisional application No. 60/612,616, filed Sep. 24, 2004 (attorney docket number 08675-6054), U.S. provisional application No. 60/615,591, filed Oct. 5, 2004 (attorney docket number 08675-6049), and U.S. Provisional application No. 60/622,112, filed Oct. 27, 2004 (attorney docket number 08675-6052), all of which are incorporated herein by reference.

This invention was made with partial Government support under contract number W911 NF-04-C-0046 awarded by DARPA. The Government has certain rights in the invention.

### BACKGROUND OF THE INVENTION

This invention relates to vaccines and treatments, as well as methods of making vaccines and treatments, to protect against the detrimental effects of biological warfare, heart diseases, cancers, and emerging infectious disease pathogens.

Viruses and bacteria are responsible for a vast number of diseases affecting humans and other animals. In addition to the pathogens encountered in everyday life, there are some pathogens that can be weaponized and used in biological warfare. These include, but are not limited to, Variola virus (smallpox virus), Bacillus anthracis, Yersinia pestis, Brucella suis, Francisella tularensis, Burkholderia mallei, encephalitis, and hemorrhagic fever viruses.

Because pathogens used in biological warfare are typically encountered in the field before they can be identified, it is important that preventative measures protect against a broad spectrum of potential pathogens. The vaccines currently available that protect against individual pathogens are, therefore, not as useful as a vaccine that would be protective against a broad spectrum of pathogens.

In addition to the pathogens that can be used in biological warfare, more commonly encountered pathogens have been found to cause some of the most prevalent diseases that debilitate humans every day. For example, cardiac disease has been tied to the involvement of microbes, such as, but not limited to, cytomegalovirus and other herpesviruses, coxsackieviruses, echoviruses, hepatitis viruses, influenza viruses, chlamydia, Streptococcus pneumoniae, and Staphylococci.

Similarly, certain cancers have also been shown to be associated with specific viruses and bacteria, including, but not limited to, human papilloma viruses, Epstein Barr virus, hepatitis B virus, human lymphotrophic viruses, retroviruses, reoviruses, human herpesvirus 8 (HHV8), influenza viruses, and coxsackieviruses, Helicobacter pylori, Citrobacter, Salmonella, etc.

Like the pathogens used in biological warfare, the pathogens related to cardiac disease and cancer cover a broad spectrum. Vaccines against individual pathogens, then, are not as effective in preventing cardiac disease or cancer as vaccines that target a correspondingly broad spectrum of pathogens.

To target a broad spectrum of pathogens, characteristics that the pathogens have in common can be considered. In fact, all viruses and bacteria produce proteolytic enzymes, called proteases, proteinases, or peptidases, terms which are used herein synonymously, which degrade proteins as part of their pathogenic mechanisms. Proteases are often involved in viral or bacterial growth in the infected host by proteolytic processing of virus structural proteins or by destroying cellular tissues.

Recently, new information about viral and bacterial proteases has become available as the genomes of more viruses and bacteria have been cloned. The genome of B. anthracis, the pathogenic agent of anthrax, has recently been sequenced (Supran et al., 2002; Read et al., 2003). Analysis of these genomic sequences will allow the identification of the proteases of these pathogens and of treatments based on these proteases.

There is a need in the art to use this newly recognized information to develop new treatments for the diseases caused by the pathogens of biological warfare, as well as for more common diseases, such as cardiac diseases, cancers and emerging infectious diseases.

### BRIEF SUMMARY OF THE INVENTION

The invention aids in fulfilling the need in the art by providing a method of developing prophylaxis or treatments for the pathogens of biological warfare, cardiac diseases, cancers, and emerging infectious diseases based on analyzing the proteases produced by the pathogens and creating a peptide or peptides that induce antibodies against the proteases in the human or other animal patient. In addition, the invention provides compositions and treatments comprising these peptides, or antibodies raised to them, which are developed using this method and which prevent the detrimental effects of biological warfare, cardiac disease, cancer, and emerging infectious diseases.

The method of the invention comprises several steps, including identifying a pathogen of interest, identifying sequences of at least one protease from the genome of the pathogen of interest, comparing the sequence of the protease or proteases with genomes of other pathogens to determine similar proteases, analyzing the proteases to determine which sequences are exposed on the surface of the protein, synthesizing a peptide or peptides corresponding to the surface sequences, determining which peptides can induce an immunogenic response in an animal, administering the immunogenic peptides to a patient or to an animal to raise antibodies, which are administered to a patient.

### BRIEF DESCRIPTION OF THE DRAWINGS

FIG. 1 depicts SDS-PAGE of B. anthracis culture supernatant (BACS) fractions separated on size exclusion column in panel A. Panels B, C, and F represent Western blots, with panel B using specific antisera a-M4EL, panel C, left, using BACS with a-M4AC, panel C, right using BACS with a-M4EP, and panel F using BACS with a-M9Coll. Zymograms are depicted in panels C and E, with the caseinolytic (C) and gelatinolytic activities of BACS. The molecular mass (KDa) of the marker proteins are indicated by arrows. In A, s denotes BACS, and numbers above correspond to column factions. In E, different amounts of BACS were loaded on a gel (15 μl, 7 μl and 3 μl, from left to right).

FIG. 2 depicts post exposure efficacy of hyperimmune rabbit sera in mice challenged with B. anthracis (Sterne). Treatment with sera and ciprofloxacin was initiated 24 hours post exposure and continued for 10 days, once daily. In panel A, 5 mg/kg of serum was administered with ciprofloxacin; in panel B, only serum was administered; in panel C, 25 mg/kg of serum was administered with ciprofloxacin.

FIG. 3 demonstrates a structural representation of chosen vaccine candidates. Structure records were available from MEROPs and the Domains database for M4, M32, and M34. M4 was represented by the structure of neutral proteinase from B. cereus. M32, which is a carboxypeptidase, was annotated in the Domains record with the structure of carboxypeptidase Apo-Yb from Pyrococcus furiosus. The structure of anthrax lethal factor was determined and represented in the MEROPs record for M34. Blink pre-computed results for M42, a glutamyl aminopeptidase, showed amino acid similarity to the structure record of aminopeptidase/glucanase from B. subtilis. PSIPRED and PredictProtein analyses were performed on the remaining peptidases M6, M9, M15, and M60, for which structure records that were similar in amino acid sequence were also displayed. For all but two peptidases, additional confirmation on surface location of the target region was depicted using the Predict Protein program. Table 2 details the structure records that represent the confirmed or inferred structural representation for each selected peptidase. The structure records were displayed using the program Rasmol (Sayle and Milner-White, 1995). The structural representation of the target region and the description of metalloprotease function (putative or experimental) are shown in the panels A through H. The highlighted region, shaded light grey, represents the alignment of the structure's amino acid residues to the peptidase amino acid residues from the target region. The dark grey residues are annotated as surface residues, according to the Rasmol program.

### DETAILED DESCRIPTION OF THE PREFERRED EMBODIMENTS

The invention encompasses a method of developing treatments for the pathogens of biological warfare, cardiac diseases, cancers, and emerging infectious diseases. In a preferred embodiment, the method is based on an analysis and comparison of the genetic sequences of protease enzymes of the responsible pathogens. In the first step of the method, a pathogen of interest is identified. Next, protease sequences from this pathogen are identified from the genome of the pathogen. Proteases that are either secreted, membrane bound, or periplasmic, which are essential for metabolism of the pathogen, are chosen for further analysis. Additionally, the proteases to be analyzed further should not have high homology to human proteases.

In the next step of the method, the genomic sequences of other pathogens are compared with the sequence(s) of the selected protease or proteases to reveal other similar proteases of pathogens likely to be involved in the same situation or disease. This search can be performed by, for example, but not limited to, a BLAST search.

Each of the identified proteases in the pool, including those of the original pathogen and those from other pathogens, is then analyzed to determine which protein sequences are exposed on the surface of the molecule and, therefore, are accessible to antibody binding. The “surface” of the molecule is any region of peptide sequence with a minimum length of 6-8 amino acids or longer that is exposed on the outside of the three-dimensional structure of the molecule. “Antibody binding” is defined as the ability of an antibody to specifically bind a peptide sequence. Specific binding is defined as greater than 10⁵ M⁻¹.

A peptide or peptides corresponding to these regions identified as being on the surface are synthesized and conjugated to immune enhancing peptide sequences. These conjugates are then administered to an animal to determine which protease peptides are antigenic and induce antibody production.

Finally, the antigenic peptide conjugates are combined to form a vaccine capable of creating a protective immune response against one or several pathogens. An “immune response” is a reaction in a host to an administered substance, wherein specific antibodies are produced or other cells of the immune system are specifically stimulated. A “protective immune response” is an immune response in which the antibodies and cellular immune reactions produced are protective against a disease or diseases. A “vaccine” is a substance administered to a human or other animal that produces an immune response and protects the human or other animal from a disease. Vaccines can be administered before or after exposure of the human or other animal to a disease.

Certain computer software can be used to predict the surface probability, antigenicity, or hydrophilicity of a stretch of amino acids residues (sequence). The MEROPs database is a curated database of peptidases and inhibitors. The data is categorized into individual summary pages of peptidases, including sequence information and organism source. Individual peptidases are then grouped into families and individual families grouped into clans (each with its own summary record), based on statistical sequence similarity (Rawlings et al., 2004). The MEROPs summary display for each peptidase family, in particular, also provides multiple alignments of the peptidase sequences within the family and a distribution chart of the peptidase family among the major taxonomic groups, including bacteria, viruses, animals, and plants (Rawlings et al., 2004).

Specific dosing ranges and methods of administration are different for various types of vaccines and therapeutic preparations. Optimal concentrations and regimens are determined empirically for each vaccine and therapeutic preparation based on prophylactic and therapeutic efficacy by dosing regimes known in the art. For example, doses of 10 μg to 1 g, 25 to 500 μg, 50 to 250 μg, 75 to 150 μg, or 30 to 50 μg can be used.

In an alternative embodiment of the invention, the antigenic peptide or peptides are administered as conjugates to an animal capable of raising large amounts of antibody, such as, but not limited to rabbits, sheep, cows, horses, mice, goats, monkeys, rats, etc. The polyclonal antibodies produced by this animal are purified and administered to a person to provide passive immunization or as a therapy for a disease. “Passive immunization” or “passive immunity” is achieved by transferring antiserum from one animal to another to achieve immunity against a disease in the recipient. Passive immunization can be performed before or after the recipient has been exposed to the disease. A “therapy” for a disease is any course of action, including the administration of substances, to a patient suffering from a disease to alleviate the symptoms of the disease.

In another alternative embodiment of the invention, the antigenic peptide or peptides are administered as conjugates to an animal and monoclonal antibodies are produced. Monoclonal antibodies can be prepared as described by Roque et al. (2004). In an embodiment of the invention, monoclonal antibodies can be engineered to be chimeric antibodies, for example, including human constant regions.

In these alternative embodiments, antibodies are administered to the person or animal in need of treatment. In one embodiment, the amount of antibody administered to the patient is from 100 mg to 1 g. In another embodiment, the amount of antibody is from 200 to 800 mg. In another embodiment, the amount of antibody is from 300 to 600 mg. In yet another embodiment, the amount of antibody is from 400 to 800 mg.

The amount of antibody can also be determined on a per weight basis. Doses of antibodies range of the invention range from 0.1 mg/kg to 100 mg/kg or more. Other embodiments include doses of 1 mg/kg to 50 mg/kg. In yet other embodiments the amount of antibody is from 5 mg/kg to 25 mg/kg. Preferably, the antibody is administered at 10 mg/kg.

In an embodiment of the invention, antibodies will be administered intravenously or subcutaneously, and antibiotics will be administered orally, intravenously, or subcutaneously. Injectable forms of the antibiotics or antibodies can be administered intravenously or subcutaneously, while oral administration can be achieved by many different methods, including but not limited to, tablets, solutions, lozenges, etc.

The invention encompasses the peptide sequences determined by the methods of the invention. In a specific embodiment, the peptide sequences useful against Bacillus anthracis include, but are not limited to: GTLHEIAHGYQA, DVIGHELTHAVT, GAVGVFAHEYGH, ELFRHEFTHYLQ, VIGHELTHAVTE, EYDTQYSGHGE, ELFRHEFTHYLQ, and SAIPGTSEHQT. The sequences that are useful against Yersinia pestis include, but are not limited to, GNMDDYDWMNEN, WVRAHDNDEHYM, MGGMAASGGYWI, LILFEPANFNSM, FNLADVAICIGML, NAGYYVTPNAKV, GRQTFTHEI, and SRHHWGSDLDI. The peptides comprising these sequences may include other sequences and, thus, be of different lengths. Preferably the peptides are less than 50, 40, 30, 20, or 15 amino acids long.

In a further embodiment of the invention, peptides are combined in an immunological composition further comprising adjuvants. Adjuvants include, but are not limited to alum, killed Bordetella pertussis, oil emulsion, Freund's complete or incomplete adjuvant, or any other material that can be added to an antigen to increase its immunogenicity. In yet further embodiments, the immunological compositions are used in vaccines.

In alternative embodiments of the invention, single pathogens are targeted or multiple pathogens are targeted with the peptide or peptides developed.

The invention further encompasses treatments for the pathogens and diseases of biological warfare, cardiac disease, cancer, and emerging infectious diseases. In these treatments, the peptide or peptides determined by the methods of the invention are administered to a patient and the effects of the disease are alleviated.

The diseases of biological warfare include, but are not limited to, anthrax, smallpox, human monkeypox, plague, tularemia, glanders, melioidosis, brucellosis, botulism, tetanus, Ebola virus infection, Marburg virus, Lassa fever, Bolivian hemorrhagic fever, Argentinean hemorrhagic fever, Venezuelan equine encephalomyelitis, etc.

Cardiac diseases include, but are not limited to, atherosclerosis, stenosis of the blood vessels, restenosis of the blood vessels, myocardial infarction, myocarditis, pericarditis, acute and chronic cardiomyopathies, hypertension, etc.

Cancers include, but are not limited to, carcinomas, leukemias, sarcomas, Burkitt's lymphoma, nasopharyngeal carcinoma, papilloma, Kaposi's sarcoma, hepatocellular carcinoma, Hodgkin's Disease, myeloid leukemia, bladder cancer, brain cancer, head and neck cancer, kidney cancer, lung cancers, such as small cell lung cancer and non-small cell lung cancer, myeloma, neuroblastoma/glioblastoma, ovarian cancer, pancreatic cancer, prostate cancer, skin cancer, liver cancer, melanoma, colon cancer, cervical carcinoma, breast cancer, epithelial cancer, and gastric cancer.

The invention encompasses treatments for the pathogens of emerging infectious diseases. These include, but are not limited to, Salmonella, Shigella, Escherichia coli, Vibrio cholerae, Haemophilus influenzae, Streptococcus pneumoniae, and Neisseria meningitides.

The present invention further relates to compositions, methods, processes, and systems for identifying and validating target proteinases and/or their specific amino acid sequences useful as therapeutic and prophylactic targets for modulating diseases associated with microbes as well as to the principles and methods of accelerated drug development. The term “microbial” includes any microorganism or microorganism-like agent, including, but not limited to, bacteria, fungi, mycoplasma, rickettsia, and viruses.

The invention further includes:

1. identifying microbial proteinases as targets for therapeutics and prophylactic preparation development;

2. identifying an amino acid sequence (or multiple amino acid sequences) as a target, which are present in a microbial proteinase;

3. identifying the common (or similar) amino acid sequences that are targets in microbial proteinases of microorganisms belonging to different species, genera, and taxonomic groups;

4. analyzing the presence of these sequences in human proteinases to prevent possible negative interactions;

5. creating libraries of target sequences from the same and different proteinases of the same microorganisms;

6. synthesizing these amino acid sequences and conjugating the resulting peptides to KLH;

7. producing the specific antibodies against these sequences in in vitro 2- or 3-D splenocyte-based tissue systems;

8. testing the neutralizing activity of the antibodies using different methods (e.g. ELISA);

9. developing single-sequence based or multi-sequence based vaccines against a single microorganism for prophylaxis, or specific immunoglobulins for both prophylaxis and therapy, of a single disease that are capable of protecting against or treating a number of diseases;

10. confirming the protective efficacy of vaccines based on the chosen sequences;

11. producing therapeutics, including either polyclonal or monoclonal antibodies, against the chosen sequences and the evaluation of the therapeutic efficacy of these antibodies.

The invention will be described in greater detail in the following Examples.

### EXAMPLE 1

### Genomic Analysis of B. anthracis Secreted Proteins as Potential Virulence Factors

A whole-genome level comparative analysis of all protease genes in the genomes of B. cereus, B. subtilis, and two virulent anthrax strains, using the known sequence motifs characteristic of hundreds of families of proteolytic enzymes, was performed. This analysis showed that the proteases that are the most potent of the ones secreted by B. anthracis in culture are metallo-protease (MP) enzymes, which act primarily as collagenases. In addition, several closely related thermolysin-like MPs of the M4 family are candidate enzymes that are capable of causing a hemorrhagic effect similar to thermolysin (EC 3.4.24.27) from Bacillus thermoproteolyticus. Moreover, these MPs are more abundant in both B. anthracis and B. cereus, compared to B. subtilis.

An analysis of the chromosome sequence of the B. anthracis Ames strain was performed based on shared sequence homology with pathogenic factors in other bacterial species. (Supran et al., 2002; Read et al., 2003) This analysis revealed a variety of potential virulence-enhancing factors, including collagenases, phospholipases, haemolysins, proteases, and other enterotoxins. In fact, the B. cereus group of bacteria, which are pathogenic to humans or insects and includes B. anthracis, B. thuringiensis, and B. cereus, has more sequences that are predicted to be secreted proteins than does nonpathogenic B. subtilis (Read et al., 2003). These B. cereus group-specific genes represent the adaptations to a pathogenic lifestyle by the common ancestor, which was quite similar to B. cereus.

The most interesting of the secreted proteins is the group of proteases encoded on the B. anthracis chromosome that are shared in common with B. cereus, but are absent or relatively rare in the genomes of nonpathogenic bacteria. A large number of these proteases fall into clan MA (classified according to the MEROPS system, Barrett A J, 2004). This clan includes thermolysin-like enzymes of the M4 family and others. The metallo-proteases (MPs) from several bacterial species belonging to this family are capable of causing massive internal hemorrhages and other life-threatening pathologies (Supuran et al., 2002; Sakata et al., 1996; Shin et al., 1996; Miyoshi et al., 1998; Okamoto et al., 1997).

Eleven protease families are present in B. anthracis and B. cereus, but absent in B. subtilis. Six of these eleven subfamilies encode MPs. Three of the MP subfamilies, namely the M6, M9B, and M20C subfamilies, are encoded on the bacterial chromosomes. Members of the M6 peptidase family are usually described as “immune inhibitors” because in B. thuringiensis they can inhibit the insect antibacterial response (Lovgren et al., 1990). The M20C peptidase subfamily represents exopeptidases (Biagini et al., 2001) that are the unlikely cause of tissue destruction or internal bleeding. The collagenolytic proteases of the M9B family have potential pathogenic functions.

This genomic analysis indicated that the M4 family of thermolysin/elastase-like neutral proteases and the M9 family of collagenases are virulence-enhancing factors of B. anthracis Ames strain.

### EXAMPLE 2

### Determination of Peptide Sequences for Multiple Pathogenic Organisms

Using the MEROPs database of peptidases, initial studies were conducted using the M4 and M9 peptidases from Bacillus anthracis, in which chemical inhibitors and rabbit immune sera against these peptidases displayed a protective efficacy in combination with antibiotic therapy. These studies suggested a multi-pathogen vaccine candidate including the M4 and M9 peptidases.

The peptidase sequences were selected based on similarity to other pathogenic organisms, as represented by their inclusion in the same peptidase family and as seen from the multiple sequence alignment that showed the sequence conservation among the peptidases. Additionally, each peptidase family was screened for noted homologs in animals (including human) based on the taxonomic distribution chart included in the MEROPs summary records. Those that did not contain any homolog in human were allowed to proceed to the next step. Thus, the resulting list of peptidase sequences encompasses vaccine candidates that can target multiple pathogenic organisms, but in which the likelihood of immune complications in humans would be minimal.

The list of peptidase sequences, which contains selected peptidase families consisting of multiple pathogenic organisms and which do not have homologs in human, is described below. Each peptidase is represented by a short amino-acid stretch that spans or is in near proximity to an active site motif. Each sequence is 6-13 amino acid residues in length, which is sufficient to allow for antibody recognition. In addition, each sequence includes the active site residue(s) as an additional mechanism to inhibit peptidase function if blocked by the immune system (Table 1).

TABLE 1

Examples of specific and common sequences of proteases and
protease homologues for vaccine and therapeutic candidates

| | | |
|---|---|---|
| Microorganism | Source | Sequence |

| | | |
|---|---|---|
| Bacillus anthracis | M4 neutral protease B | VIGHELTHAVTE |
| | M6 Immune inhibitor A | EYDTQYSGHGE |
| | M9 Collagenase | ELFRHEFTHYLQ |
| | M15 VanY, D-alanyl-D-alanine carboxypeptidase | SAIPGTSEHQT |
| | M32 Unassigned | FGTIHECGHAVY |
| | M34 Lethal factor precursor (LF) (Anthrax lethal toxin | EGFIHEFGHAVD |
| | endopeptidase component) | |
| | M42 Glutamyl aminopeptidase | EEVGLRGAKTS |
| | M50 Unassigned | LVSIAGPISN |
| | M60 Enhancin | WGTLHEIAHGYQAG |
| | C50 Sortase | HRMKDGSMFGS |
| | S15 Putative Xaa-Pro dipeptidyl-peptidase | VHGLNDWNVK |
| | | WLHQGGHGG |
| | Edema Factor | EIDNGKKYYLL |
| | Anthrax Protective Antigen | YNDKLPLYIS |
| | M4 | DVIGHELTHAVTE |
| | M6 | DGAVGVFAHEYGH |
| | M32 | MGIVHETGHARYE |
| | M34 | EGFIHEFGHAVDD |
| | M42 | VAGHLDEVGFMI |
| Yersinia pestis | A8 Signal peptidase | FNLADVAICIGAAL |
| | A26 Plasminogen activator | NAGYYVTPNAKV |
| | M10 Secreted metalloprotease | GRQTFTHEI |
| | | GHALGLSHP |
| | M15 YPO3054 protein | SRHHWGSDLDI |
| | M32 putative carboxypeptidase | LMGIVHETGHARYE |
| | C55 YopJ endopeptidase | EMDIQRSSSECGIFS |
| | C58 Unassigned | SCEGSQFKLFD |
| | S49 Peptidase IV | SMGGMAASGGYVVIS |
| | U62 Modulator of DNA gyrase | AKGYWVENGEI |
| | V antigen | TTIQVDGSEKK |
| | F1 capsule antigen | SQDGNNHQFTT |
| | Murine toxin | DPQWKYSQETA |
| | YopD secreted effector protein | DNFMKDVLRLIEQ |
| | | EQYVSSHTHAMK |
| | YopH phosphatase (B chain) | SPYGPEARAELSSR |
| | A26 Plasminogen activator | GNMDDYDWMNEN |
| | A26 plasminogen activator | WVRAHDNDEHYM |
| | A8 signal peptidase | FNLADVAICIGAAL |
| | M15 YPO3054 protein | SRHHWGSDLDIYDP |
| | M60 unassigned | WGCLHEIAHGYQGGF |
| | C55 YopJ endopeptidase | LILFEPANFNSMG |
| Francisella tularensis | A24 Type IV pili leader peptidase and methylase | GYGDFKLLAA |
| | C59 Choloylglycine hydrolase family protein | KPQFTSYSVVD |
| | M16 Unassigned | DELNSIVENN |
| | M20 Aminoacylase | TSMVHEPNFD |
| | M22 O-sialoglycoprotein endopeptidase | EGHLLSPLLD |
| | M41 ATP dependent metalloprotease | TAYHEAGHAI |
| | S 11 D-alanyl-D-alanine carboxypeptidase | STGGSKMYVK |
| | S13 D-alanyl-D-alanine carboxypeptidase | NRLMTPASTN |
| | S14 CIpP protease | ETIVKDTDRD |
| | S49 Unassigned | YWFGKDALEL |
| | T3 Gamma-glutamyltranspeptidase | EKLQTTHFSI |
| | U32 Protease YegQ | IHLSVQANAV |
| | MviN virulence factor | GKFSLDVDFT |
| | Bacterioferritin | THFGEHPSLKI |
| | AcpA Acid Phosphatase | DAMSTNKFGV |
| | IspH | VGSQNSSNSNR |
| Variola Major | Major secreted protein, chemokine binding protein | SSVSPGQGKDSP |
| | | GSNISHKKVSYED |
| | Protein E3 | LYDLQRSAMVY |
| | K3L | YRDKLVGKTVK |
| | VP39 | PSTADLLSNY |
| | | LKWRCPFPDQ |
| | Protein K5 | LSYSREQT |
| | M44 endopeptidases | LGIAHLLEHLLISF |
| | M44 endopeptidase | IRFHIKELENEYYF |
| | C55 17L processing peptidase | MFGFCYLSHWKCVI |
| | | CLVSFYDSGGNIPT |
| | | VEVNQLLESECGMFI |
| Clostridium botulinum | M27 Botulinum neurotoxin type A precursor | LAHELIHAGHRL |
| | | FEELRTFGGHD |
| | | YNQYTEEEKNNI |
| | | HQFNNIAKL |
| | | VASNWYNRQ |
| | | IERSSRTLG |
| | M27 Botulinum neurotoxin type B precursor | LMHELIHVLHGL |
| | | AEELYTFGGQD |
| | | YNIYSEKEKSNI |
| | | HRFYESGIVFEEY |
| | | KDYFCISKWYLK |
| | | EVKRKPYNLKLG |
| | M27 Neurotoxin [Clostridium botulinum C] | LMHELNHTMHNL |
| | | YAEIYAFGGPT |
| | | YKKYSGSDKENI |
| | M27 Botulinum neurotoxin type D precursor | LMHELTHSLHQL |
| | | FEELYTFGGLD |
| | | YKKYSGSDKENI |
| | M27 Botulinum neurotoxin type E precursor | LMHELIHSLHGL |
| | | IEEFLTFGGTD |
| | | YNSYTLEEKNEL |
| | | STWYYTHMRDH |
| | M27 Botulinum neurotoxin type F precursor | LAHELIHALHGL |
| | | LEEFLTFGGQD |
| | | YNNYTSDEKNRL |
| | | HSNNLVASSWY |
| | | YNNIRRNTSSNG |
| | M27 Botulinum neurotoxin type G precursor | LMHELIHVLHGL |
| | | AEELYIFGGHD |
| | | YNRYSEEDKMNI |
| | | SQWYLRRIS |
| | | ENINKLRLG |
| | C2 toxin (component-I) | WGKEEEKRW |
| | | PIPETLIAYRR |
| | C2 toxin (component-II) | DTDRDGIPDEWE |
| | | GQIDPSVS |
| Shigella sp. | A8 Leader peptidase II | DRLWHGFWD |
| | | WHFATFNLAD |
| | A24 type IV prepilin peptidase 2 | GYGDVKFLAA |
| | A26 IcsA-cleaving protein | SMVDKDWNNS |
| | | SDTDKHYQTE |
| | M15 Unassigned | NNLHTGESIK |
| | M16 Pitrilysin | HEKNVMNDAW |
| | M20 N-succinyl-diaminopimelate deacylase | HTDVVPPGDA |
| | | EEASAHNGTV |
| | | NATIHKINEC |
| | M20 Peptidase T | YNYHGKHEF |
| | | HVDTSPDCSG |
| | | EEVGKGAKHF |
| | | DGGGVGELEF |
| | M20 Xaa-His dipeptidase | TMTEEAGMDGA |
| | | GPTITGPHSP |
| | M74 Murein endopeptidase | GGRFNGGHASH |
| Bacillus anthracis/ | M16 Unassigned/protease III | HFLEHM/HYLEHM |
| Yersinia pestis | | |
| | M20C Unassigned/aminoacyl-histidine dipeptidase | TTLGADNGI |
| | | GLKGGHSG |
| | | NAIPRE |
| | M23B/M37 Unassigned | GQKVKQG/GQKVKRG |
| | M32 Unassigned/putative carboxypeptidase | IHECGHAVYEQNI/ |
| | | VHETGHARYEQNL |
| | | IHESQSL |
| | | RITTRY |
| | M60 Enhancin | LHEIAHGYQ |
| | S1 Trypsin/Periplasmic secreted protease Do | GNSGGAL |
| | | EGIGLAIP/IGIGFAIP |
| | S11 D-alanyl-D-alanine carboxypeptidase; penicillin-binding | PASMTKIM/PASLTKIM |
| | protein 6 | |
| | | SGNDAS |
| | | MRVISW/MRLISW |
| | S26 Signal peptidase I | SMMPTL |
| | | DYVKRIIGLPGD/ |
| | | DYIKRVVGLPGD |
| | S33 hydrolase/proline iminopeptidase | FVHGGPGIFIHGGPG |
| | T3 Gamma-glutamyl-transpeptidase | GFFLNN |
| | | GSPGGNRI/GSPGGSRI |
| | U32 Unassigned | AYSGRC |
| | | SLKVEGR/SLKIEGR |
| | oligopeptide abc transporter/putative substrate-binding | LKFSDGSPLTA |
| | periplasmic protein of ABC transporter | |
| | glycerol-3-phosphate-binding periplasmic protein | KAGLDPE |
| | ribose ABC transporter/solute-binding periplasmic protein of | AHNDEMALGA/ |
| | ABC transporte | AHNDDMAIGA |
| | iron compound abc transporter/putative iron-siderophore | LKPDLII/LKPDVII |
| | transport system | |
| | molybdate-binding periplasmic protein | QIEQGAPADLF |

The italicized amino acids represent the active site residue, and the bolded amino acids represent metal-binding residues.

Each peptidase sequence contains a list of organisms that have similar sequences to the selected peptidase, either as curated by the MEROPs database or retrieved from the NCBI Blink program, which is a pre-computed form of BLAST (Altschul et al., 1997), using the default parameters. The resulting collection of sequences, from multiple organisms that represent sequence similarity to the selected peptidase, was put into a multiple alignment program called MultAlin (Corept, 1988) to verify sequence conservation among sequences in each group, especially in the region of the active site residue(s). The sequences were then compared by a BLAST search against the human Reference Sequence (RefSeq) collection of proteins (Pruitt et al., 2003) to ensure that they do not express significant homology to any human protein.

### EXAMPLE 3

### Determination of 3-Dimentional Structure of Chosen Vaccine Candidates

The MEROPs database offers a wealth of information on Bacillus peptidases, including sequence data, catalytic activity, active-site residues, known structural conformations (when available) and relevant literature pertaining to function. It is known that a large number of peptidases exist that are potential virulence or virulence enhancing factors of bacteria and can be used as promising targets for vaccine and antibody development. The group of metalloproteinases that are encoded on the Bacillus anthracis chromosome and plasmids, and are not over-represented in non-pathogenic bacteria were highlighted because many of them play a significant role in the pathogenesis of many bacterial infections. For example, some metalloproteinases of the MA clan from several bacterial species, such as the M4 and M9 families, are known to contribute in life-threatening pathologies (Miyoshi et al., 1998; Okamoto et al., 1997; Sakata et al., 1996; Shin et al., 1996; Supuran et al., 2002). The MA clan's M6 metalloproteinase is a known immune inhibitor in some other Bacillus species and M34 metalloproteinase is the lethal factor of Bacillus anthracis.

A metalloproteinase selection process began by screening all 85 subfamilies of metalloproteinases and selecting eight peptidases by using the following selection principles and methods. First, the taxonomic distribution chart for each Bacillus anthracis metallopeptidase family was screened and metallopeptidases that did not contain homologues in animals (including human) were chosen. Thus, immune complications as a result of cross-reactivity to similar human proteins were avoided. Additionally, the selected peptidase sequences were BLASTed against the human Reference Sequence (RefSeq) collection of proteins (Pruitt et al., 2003) to confirm that they do not express significant homology to any human protein (Altschul et al., 1997). Default parameters were used and results were scanned based on a threshold of 25% identity if the selected peptidase matched a human protein. From this analysis, no selected peptidase sequence showed a significant similarity above the identity threshold to any human protein.

Combined with the above computational analysis performed for a potential target peptidase, the existing literature was reviewed and peptidases that are known to play a role in anthrax pathogenesis, such as M34 (anthrax lethal factor), or those that possess convincing evidence of their role in pathogenesis were chosen. In addition, the localization of the peptidase in relation to the bacterial cell were studied and peptidases that were either membrane-bound or extracellular were chosen, such that these peptidases can be easily accessible to the human immune system. Thus, from numerous Bacillus anthracis's metalloproteinases in the MEROPs database, several peptidases were identified to be potential targets designated for further analysis.

A structural analysis of the selected peptidase sequences was then performed to determine the tertiary conformation of the peptidase. In particular, the location of the active site region in each selected peptidase was noted because it would serve as the best region to be targeted by the immune system (e.g., annihilation of peptidase functions if blocked by an antibody). For each peptidase, a target region that consisted of from six to thirteen amino-acids that spans the active site motif or is within proximity of the active site motif, which is sufficient to allow for antibody recognition, was considered. In addition, localization of the peptidase target regions was checked, for any residue within the region, to see whether it is placed on the surface of the peptidase. This was performed to clarify whether the target region would be accessible to the outside and capable of interacting with the human immune system and mediating specific immune response.

Structure data from the MEROPs records (if they were available) for any of the selected peptidases were collected. In cases where the MEROPs database could not provide a structural representation of a selected peptidase sequence, a conserved domain (CDD) search was performed using RPS-BLAST (default parameters, Marchler-Bauer et al., 2004), and structural representations were presented when available from a given Domain record (Marchler-Bauer et al, 2003). If no structure representations were available for a peptidase of interest in both the MEROPs database and the Domains database, then a Blink search (pre-computed with default parameters) was performed for structure records that showed amino acid sequence similarity to the selected peptidase sequence (Wheeler et al., 2004). If a similar structure record was found in Blink, then the structure record was visually displayed along with its amino acid region aligned to the selected peptidase sequence, thereby, inferring the structural configuration of the selected peptidase.

When structure records were not found from any of the above methods for a peptidase sequence, then the selected peptidase sequence was used in structure prediction analysis tools that allocate predicted chemical characteristics, such as solvent accessibility plots, and/or show similarity to existing structure records. Two such programs were used for this purpose: PredictProtein and PSIPRED. The PredictProtein program was used to detect chemical properties attributable to the selected peptidases, such as solvent accessibility (which infers localization of the sequence to the outside region of the protein) (Rost et al., 1996, and Rost et al., 1994). The PSIPRED protein structure prediction server provides structure predictions based on one of three methods; the one that was used in this case was GenTHREADER, a sequence profile based fold recognition method (McGuffin et al., 2000).

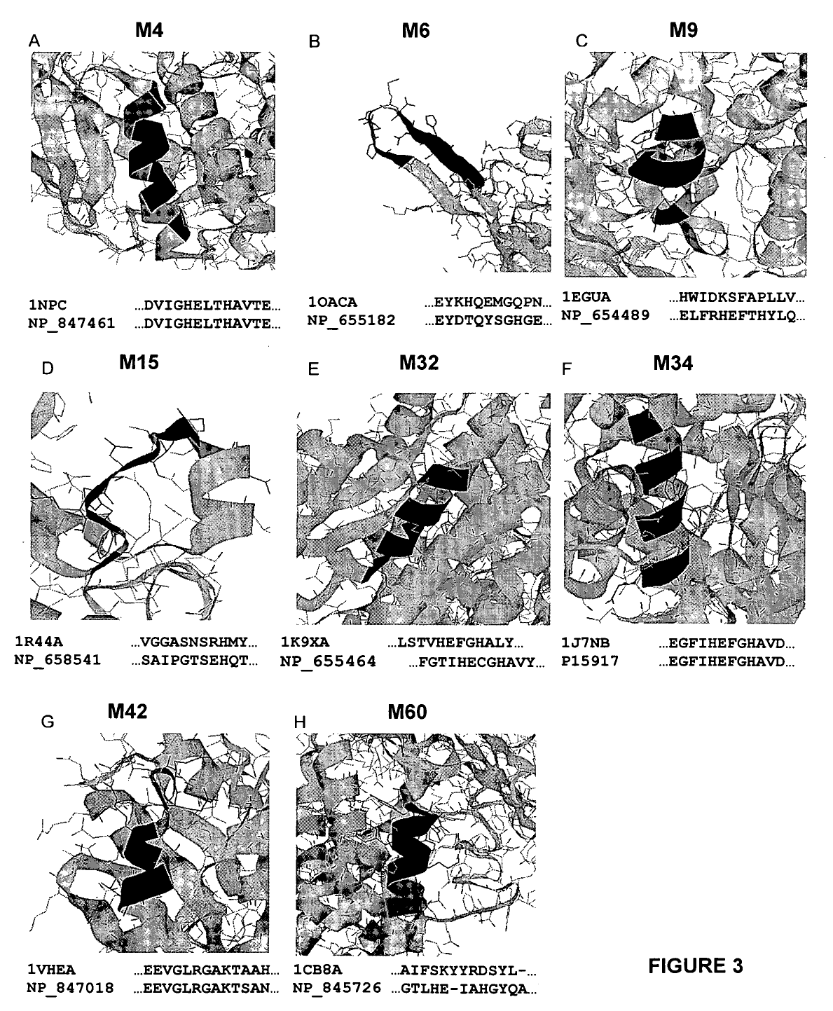

Next, for each selected peptidase sequence from the group of potential targets, structure data was retrieved from curated databases and pre-computed analysis. In addition, structure data was retrieved from structure prediction tools, in order to deduce the 3D conformation of the peptidase and infer the placement of the amino acid residues—specifically the target region encompassing the active site residues—relative to the peptidase (FIG. 3).

Table 2 highlights the selected metalloproteinases of Bacillus anthracis and selected specific peptide sequences of these metalloproteinases and a short description of the computational approach used to select them. As is evident, each chosen metalloproteinase shows no significant homology to any human protein. There is evidence of a presumed role in anthrax pathogenesis. Each target sequence region is determined or predicted to be localized to the outside of the protein and the bacterial cell, which is important for human immune recognition. The structural representations for each peptidase are listed, with reference to the source used to derive the structure records; and where suitable, identity scores are shown to reflect the significance of the amino acid similarity between a structure record and the peptidase sequence.

TABLE 2
Sequences of vaccine candidates for Bacillus anthracis derived from initial
bioinformatics screening.

| PEPTIDASE NAME | SEQUENCE/ | BLAST RESULTS (VS. | |
|---|---|---|---|
| (MEROPS | PROTEIN ACCESSION | HUMAN REFSEQ | STRUCTURE |
| DATABASE) | (ID) | PROTEINS) | RESULTS |
| M4 thermolysin family | DVIGHELTHAVTE | No significant similarity | MEROPs-1NPC |
| | NP_847461 | | |
| M6 immune inhibitor A | EYDTQYSGHGE | No significant similarity | PSIPRED-1OACA |
| family | NP_655182 | | E value = .035 |
| | | | Net score = .624 |
| | | | Predict Protein |
| | | | shows region of high |
| | | | solvent accessibility |
| M9 peptidase family | ELFRHEFTHYLQ | No match to region of | PSIPRED-1EGUA |
| (bacterial collagenase) | NP_654489 | interest | E value = .001 |
| | | | Net score = .794 |
| | | | Predict Protein |
| | | | shows partial region |
| | | | of high solvent |
| | | | accessibility |
| M15 D-Ala-D-Ala | SAIPGTSEHQT | No significant similarity | PSIPRED-1R44A |
| carboxypeptidase | NP_658541 | | E value = .002 |
| | | | Net score = .766 |
| | | | Predict Protein |
| | | | shows partial region |
| | | | of high solvent |
| | | | accessibility |
| M32 carboxypeptidase | FGTIHECGHAVY | No significant similarity | Domains-1K9XA |
| Taq | NP_655464 | | |
| M34 anthrax lethal | EGFIHEFGHAVD | Match covering region | MEROPs-1J7NB |
| factor family | P15917 | of interest is 19% | Predict Protein |
| | | identical (less than | shows partial region |
| | | required 25%) | of high solvent |
| | | | accessibility |
| M42 glutamyl | EEVGLRGAKTSAN | No significant similarity | Blink-1VHEA |
| aminopeptidase family | NP_847018 | | Score = 1467 |
| | | | Identity = 99% |
| | | | Predict Protein |
| | | | shows partial region |
| | | | of high solvent |
| | | | accessibility |
| M60 enhancin family | GTLHEIAHGYQA | No match to region of | PSIPRED-1CB8A |
| | NP_845726 | interest | E value = .547 |
| | | | Net score = .175 |
| | | | Predict Protein |
| | | | shows partial region |
| | | | of high solvent |
| | | | accessibility |

### EXAMPLE 4

### Generation of Antibodies Against B. anthracis MPs

Obvious complexity of the B. anthracis culture supernatant (“BACS”) protein composition required the development of specific means of detection and inhibition of its components. Accordingly, several immune sera were raised in mice and rabbits using the antigens listed in Table 3. The sera were used in Western blots of BACS proteins.

TABLE 3
Sera against B. anthracis proteases

| | Protein | | Gene | | |
|---|---|---|---|---|---|
| Serum # | family | Protein | number | Antigen | Designation |
| 1 | M4 | Elastase-like | BA3442 | Recombinant | M4EL |
| | | neutral protease | | polypeptide | |
| | | | | corresponding to the | |
| | | | | fragment 248-532. | |
| 2 | M9 | Collagenase | BA0555, | HEFTHYLQGRYEVPGL | M9Coll |
| | | | BA3299, | spanning the region of | |
| | | | BA3584 | active center | |
| 3 | M4 | Neutral protease | BA5282, | DVIGHELTHAVTE | M4AC |
| | | | BA0599 | spanning the region of | |
| | | | | active center | |
| 4 | M4 | Neutral protease | BA2730 | ADYTRGQGIETY | M4EP |
| | | | | distant from the active | |
| | | | | center | |

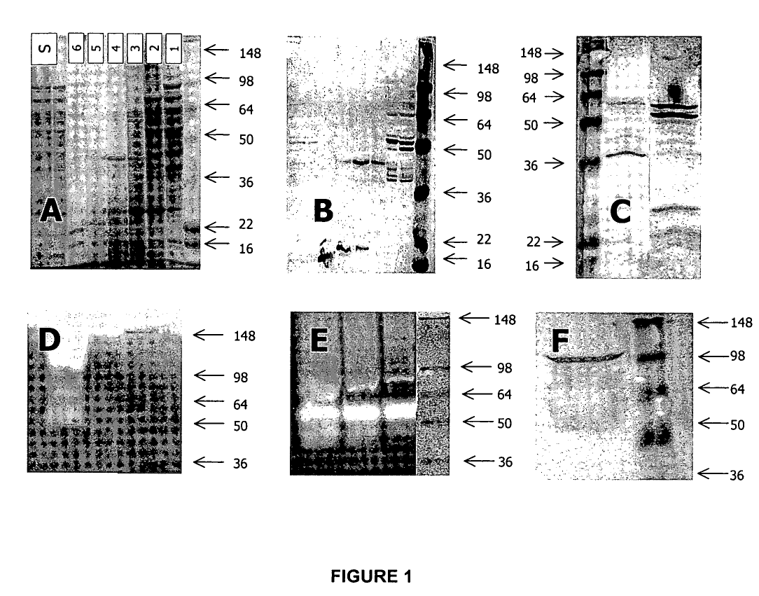

When the proteins were directly separated in the SDS-PAGE for subsequent transfer to the nitrocellulose membrane, the resulting blots were of low intensity, indicating that proteolytic degradation had occurred during electrophoresis (FIG. 1A, left lane). In order to avoid this complication, the BACS was fractionated according to the molecular masses of its components on the Superdex size exclusion column in the presence of EDTA as a chelating agent.

Analysis of the column fractions in SDS-PAGE showed a complex pattern of proteins bands (FIG. 1). Multiple proteins with a broad spectrum of molecular masses seem to be highly associated and migrate through the column as high molecular mass complexes. Several factors, such as the presence of multiple precursor and mature protein forms resulting from specific proteolytic maturation, along with nonspecific proteolytic products, can potentially contribute to the complexity of the composition of the fractions. Western blot experiments with column fractions revealed several discrete bands recognized by antibodies (FIG. 1). The M4 proteases are represented by several bands at about 50 KDa, as well as by the bands at about 40 and 20 KDa. These bands probably correspond to different maturation forms of proteases, including the enzymes lacking signal peptides, and mature enzyme forms. The M9 collagenases are detected as a band with a molecular mass of about 98 kDa, which is close to the estimated mass of the pro-enzymes, however the major gelatinase enzymatic activity corresponds to the 55 kDa proteins in the BACS.

### EXAMPLE 5

### Protection of Mice Against B. anthracis Using Anti-Protease Sera

In order to evaluate a pathogenic potential attributed to the B. anthracis proteins other than known lethal and edema toxins, a nontoxigenic and nonencapsulated strain of B. anthracis (delta Ames), which is a parental Ames strain cured of both plasmids, pXO1 and pXO2, was used.

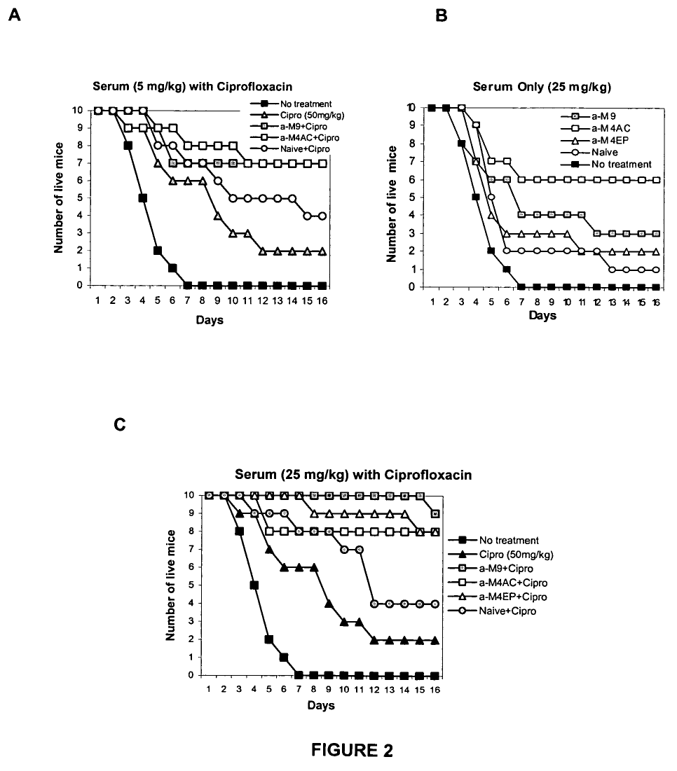

Mice were challenged intraperotineally (i.p.) with about 30 LD50 of B. anthracis Sterne spores. Treatment with a single daily dose of ciprofloxacin (50 mg/kg, i.p.) began at 24 h post challenge and continued for 10 days. The immune sera (each pulled from two rabbits) were administered once daily at a concentration of 25 mg/ml (i.p.). The sera displayed substantial differences in their protective effect (FIG. 2). The anti-M4 serum against the epitope(s) of the active center displayed the highest protection (60%), while the anti-collagenase serum (a-M9Coll) protected 30% mice. The anti-M4EP serum behaved similar to the naïve serum. Both latter sera demonstrated no statistically reliable difference in survival, compared to untreated mice (10%, p>0.05). A combination treatment with both antibiotic and all studied immune sera, administered at the same dose (25 mg/kg) was synergistic and protected from 80 to 100% mice. A lower serum dose (5 mg/kg) showed a similar pattern of protection, however the effect of combination treatment was reduced to 70%.

The sequence of the M4EP peptide is ADYTRGQGIETY. The sequence of the M4AC peptide is DVIGHELTHAVTE. The sequence of the M9Coll peptide is HEFTHYLQGRYEVPGL

### REFERENCES

The following references are cited herein. The entire disclosure of each reference is relied upon and incorporated by reference herein.
1. Altschul S F, Madden T L, Schaffer A A, Zhang J, Zhang Z, Miller W, Lipman D J. Gapped BLAST and PSI-BLAST: a new generation of protein database search programs. Nucleic Acids Res. 1997 Sep. 1; 25(17):3389-402.
2. Barrett A. J. Bioinformatics of proteases in the MEROPS database. 2004. Curr. Opin. Drug Discov. Devel. 7(3):334-41.
3. Biagini A., and A. Puigserver. 2001. Sequence analysis of the aminoacylase-1 family. A new proposed signature for metalloexopeptidases. Comp. Biochem. Physiol. B. Biochem. Mol. Biol. 128(3):469-81.
4. Corpet F. Multiple sequence alignment with hierarchical clustering. Nucleic Acids Res. 1988 16(22):10881-10890.
5. Lovgren A., M. Zhang, A. Engstrom, G. Dalhammar, and R. Landen. 1990. Molecular characterization of immune inhibitor A, a secreted virulence protease from Bacillus thuringiensis. Mol. Microbiol. 4(12):2137-46.
6. Marchler-Bauer A, Bryant S H. CD-Search: protein domain annotations on the fly. Nucleic Acids Res. 2004 Jul. 1; 32(Web Server issue):W327-31. Marchler-Bauer A, Anderson J B, DeWeese-Scott C, Fedorova N D, Geer L Y, He S, Hurwitz D I, Jackson J D, Jacobs A R, Lanczycki C J, Liebert C A, Liu C, Madej T, Marchler G H, Mazumder R, Nikolskaya A N, Panchenko A R, Rao B S, Shoemaker B A, Simonyan V, Song J S, Thiessen P A, Vasudevan S, Wang Y, Yamashita R A, Yin J J, Bryant S H. CDD: a curated Entrez database of conserved domain alignments. Nucleic Acids Res. 2003 Jan. 1; 31 (1):383-7.
7. Marchler-Bauer A, Bryant S H. 2004. CD-Search: protein domain annotations on the fly. Nucleic Acids Res. Jul. 1; 32(Web Server issue):W327-31.
8. Miyoshi S., H. Nakazawa, K. Kawata, K. Tomochika, K. To be, and S. Shinoda. 1998. Characterization of the hemorrhagic reaction caused by Vibrio vulnificus metalloprotease, a member of the thermolysin family. Infect. Immun. 66(10):4851-5.
9. McGuffin L J, Bryson K, Jones D T. The PSIPRED protein structure prediction server. Bioinformatics. 2000 April;16(4):404-5.
10. Okamoto T., T. Akaike, M. Suga, S. Tanase, H. Horie, S. Miyajima, M. Ando, Y. Ichinose, and H Maeda. 1997. Activation of human matrix metalloproteinases by various bacterial proteinases. J. Biol. Chem. 28;272(9):6059-66.
11. Pruitt K D, Tatusova T, Maglott D R. NCBI Reference Sequence project: update and current status. Nucleic Acids Res. 2003 Jan. 1; 31 (1):34-7.
12. Rawlings, N. D., Tolle, D. P. & Barrett, A. J. (2004) MEROPS: the peptidase database. Nucleic Acids Res. 32 Database issue, D160-D164.
13. Read T. D., S. N. Peterson, N. Tourasse, L. W. Baillie, I. T. Paulsen, K. E. Nelson, et al. 2003. The genome sequence of Bacillus anthracis Ames and comparison to closely related bacteria. Nature. 423(6935):81-6.
14. Roque A C, Lowe C R, Taipa M A 2004 Antibodies and Genetically engineered related molecules: production and purification. Biotechnol. Prog. 20(3):639-54.
15. Rost B. PHD: predicting one-dimensional protein structure by profile based neural networks. Meth. in Enzym., 1996, 266, 525-539.
16. Rost B & Sander C. Conservation and prediction of solvent accessibility in protein families. Proteins, 1994, 20, 216-226.
17. Sakata Y., T. Akaike, M. Suga, S. Ijiri, M. Ando, and H. Maeda 1996. Bradykinin generation triggered by Pseudomonas proteases facilitates invasion of the systemic circulation by Pseudomonas aeruginosa. Microbiol. Immunol. 40(6):415-23.
18. Shin Y. H., T. Akaike, M. M. Khan, Y. Sakata, and H. Maeda 1996. Further evidence of bradykinin involvement in septic shock: reduction of kinin production in vivo and improved survival in rats by use of polymer tailored SBTI with longer t1/2. Immunopharmacology. 33(1-3):369-73.
19. Supuran C. T., A. Scozzafava, and B. W. Clare 2002. Bacterial protease inhibitors. Med. Res. Rew. 22(4):329-72.
20. Wheeler D L, Church D M, Federhen S, Lash A E, Madden T L, Pontius J U, Schuler, G D, Schriml L M, Sequeira E, Tatusova T A, Wagner L. Database resources of the National Center for Biotechnology. Nucleic Acids Res. 2003 Jan. 1; 31 (1):28-33.

1. A method of treating a patient infected with a virus, wherein the method comprises administering a treatment comprising IFN-α, IFN-γ, or IFN-γ in a combination with ribavirin to the patient
wherein the virus is selected from the group consisting of pox-type viruses and viruses that cause common cold.

2. A method of protecting a patient against an infection caused by a virus, wherein the method comprises administering a preparation comprising IFN-α, IFN-γ, or IFN-γ in a combination with ribavirin to the patient before the patient is exposed to the virus as prophylaxis, wherein the virus is selected from the group consisting of pox-type viruses and viruses that cause common cold.

3. The method as claimed in claim 1, wherein the treatment is administered within 48 hours after the patient was exposed to the virus.

4. The method as claimed in claim 1, wherein the treatment is administered intranasally.

5. The method as claimed in claim 1, wherein the IFN-α or IFN-γ is recombinant, liposome encapsulated, pegylated, or in native, isolated form.

6. The method as claimed in claim 1, wherein the treatment further comprises a pharmaceutical carrier.

7. The method of claim 1, wherein a pox-type virus is selected from the group consisting of variola, monkey poxvirus, vaccinia, and molluscipox virus.

8. The method of claim 1, wherein a viruses that causes common cold is selected from the group consisting of rhinoviruses, adenoviruses, enteroviruses, and respiratory syncytial virus (RSV).

9. The method of claim 1, wherein the administering of the treatment or vaccine alleviates or prevents at least one effect of the infection.

10. The method of claim 9, wherein the effect of the infection is selected from the group consisting of death, viral load, fever, pneumonia, edema, malaise, headache, backache, skin lesions, mucous membrane lesions, bleeding, and dehydration.

11. The method of claim 1, wherein ribavirin and IFN-γ are admixed and administered simultaneously.

12. The method of claim 1, wherein ribavirin and IFN-γ are administered sequentially.

13. A method of minimizing or preventing adverse side effects of a vaccine against a pox-type virus in a patient, wherein the method comprises administering an interferon in a combination with ribavirin to the patient.

14. The method of claim 13, wherein the pox-type virus is smallpox.

15. The method of claim 13, wherein the patient suffers for a pre-existing condition selected from a group consisting of deficient immune system, eczema.

16. The method of claim 13, wherein interferon is selected from the group consisting of IFN-α, IFN-β, and IFN-γ.

17. The method as claimed in claim 2, wherein the IFN-α or IFN-γ is recombinant, liposome encapsulated, pegylated, or in native, isolated form.

18. The method of claim 2, wherein a pox-type virus is selected from the group consisting of variola, monkey poxvirus, vaccinia, and molluscipox virus.

19. The method of claim 2, wherein a viruses that causes common cold is selected from the group consisting of rhinoviruses, adenoviruses, enteroviruses, and respiratory syncytial virus (RSV).

20. The method of claim 2, wherein ribavirin and IFN-γ are admixed and administered simultaneously.

21. The method of claim 2, wherein ribavirin and IFN-γ are administered sequentially.


SEQUENCE LISTING (187 sequences)

SEQ ID NO: 1 - protein, 12 aa, Artificial
  chemically synthesized peptide
GTLHEIAHGYQA

SEQ ID NO: 2 - protein, 12 aa, Artificial
  chemically synthesized peptide
DVIGHELTHAVT

SEQ ID NO: 3 - protein, 12 aa, Artificial
  chemically synthesized peptide
GAVGVFAHEYGH

SEQ ID NO: 4 - protein, 12 aa, Artificial
  chemically synthesized peptide
ELFRHEFTHYLQ

SEQ ID NO: 5 - protein, 12 aa, Artificial
  chemically synthesized peptide
VIGHELTHAVTE

SEQ ID NO: 6 - protein, 11 aa, Artificial
  chemically synthesized peptide
EYDTQYSGHGE

SEQ ID NO: 7 - protein, 12 aa, Artificial
  chemically synthesized peptide
ELFRHEFTHYLQ

SEQ ID NO: 8 - protein, 11 aa, Artificial
  chemically synthesized peptide
SAIPGTSEHQT

SEQ ID NO: 9 - protein, 12 aa, Artificial
  chemically synthesized peptide
GNMDDYDWMNEN

SEQ ID NO: 10 - protein, 12 aa, Artificial
  chemically synthesized peptide
WVRAHDNDEHYM

SEQ ID NO: 11 - protein, 12 aa, Artificial
  chemically synthesized peptide
MGGMAASGGYWI

SEQ ID NO: 12 - protein, 12 aa, Artificial
  chemically synthesized peptide
LILFEPANFNSM

SEQ ID NO: 13 - protein, 14 aa, Artificial
  chemically synthesized peptide
FNLADVAICIGAAL

SEQ ID NO: 14 - protein, 12 aa, Artificial
  chemically synthesized peptide
NAGYYVTPNAKV

SEQ ID NO: 15 - protein, 9 aa, Artificial
  chemically synthesized peptide
GRQTFTHEI

SEQ ID NO: 16 - protein, 11 aa, Artificial
  chemically synthesized peptide
SRHHWGSDLDI

SEQ ID NO: 17 - protein, 12 aa, Artificial
  chemically synthesized peptide
VIGHELTHAVTE

SEQ ID NO: 18 - protein, 11 aa, Artificial
  chemically synthesized peptide
EYDTQYSGHGE

SEQ ID NO: 19 - protein, 12 aa, Artificial
  chemically synthesized peptide
ELFRHEFTHYLQ

SEQ ID NO: 20 - protein, 11 aa, Artificial
  chemically synthesized peptide
SAIPGTSEHQT

SEQ ID NO: 21 - protein, 12 aa, Artificial
  chemically synthesized peptide
FGTIHECGHAVY

SEQ ID NO: 22 - protein, 12 aa, Artificial
  chemically synthesized peptide
EGFIHEFGHAVD

SEQ ID NO: 23 - protein, 11 aa, Artificial
  chemically synthesized peptide
EEVGLRGAKTS

SEQ ID NO: 24 - protein, 10 aa, Artificial
  chemically synthesized peptide
LVSIAGPISN

SEQ ID NO: 25 - protein, 14 aa, Artificial
  chemically synthesized peptide
WGTLHEIAHGYQAG

SEQ ID NO: 26 - protein, 11 aa, Artificial
  chemically synthesized peptide
HRMKDGSMFGS

SEQ ID NO: 27 - protein, 10 aa, Artificial
  chemically synthesized peptide
VHGLNDWNVK

SEQ ID NO: 28 - protein, 9 aa, Artificial
  chemically synthesized peptide
WLHQGGHGG

SEQ ID NO: 29 - protein, 11 aa, Artificial
  chemically synthesized peptide
EIDNGKKYYLL

SEQ ID NO: 30 - protein, 10 aa, Artificial
  chemically synthesized peptide
YNDKLPLYIS

SEQ ID NO: 31 - protein, 13 aa, Artificial
  chemically synthesized peptide
DVIGHELTHAVTE

SEQ ID NO: 32 - protein, 13 aa, Artificial
  chemically synthesized peptide
DGAVGVFAHEYGH

SEQ ID NO: 33 - protein, 13 aa, Artificial
  chemically synthesized peptide
MGIVHETGHARYE

SEQ ID NO: 34 - protein, 13 aa, Artificial
  chemically synthesized peptide
EGFIHEFGHAVDD

SEQ ID NO: 35 - protein, 12 aa, Artificial
  chemically synthesized peptide
VAGHLDEVGFMI

SEQ ID NO: 36 - protein, 14 aa, Artificial
  chemically synthesized peptide
FNLADVAICIGAAL

SEQ ID NO: 37 - protein, 12 aa, Artificial
  chemically synthesized peptide
NAGYYVTPNAKV

SEQ ID NO: 38 - protein, 9 aa, Artificial
  chemically synthesized peptide
GRQTFTHEI

SEQ ID NO: 39 - protein, 9 aa, Artificial
  chemically synthesized peptide
GHALGLSHP

SEQ ID NO: 40 - protein, 11 aa, Artificial
  chemically synthesized peptide
SRHHWGSDLDI

SEQ ID NO: 41 - protein, 14 aa, Artificial
  chemically synthesized peptide
LMGIVHETGHARYE

SEQ ID NO: 42 - protein, 15 aa, Artificial
  chemically synthesized peptide
EMDIQRSSSECGIFS

SEQ ID NO: 43 - protein, 11 aa, Artificial
  chemically synthesized peptide
SCEGSQFKLFD

SEQ ID NO: 44 - protein, 14 aa, Artificial
  chemically synthesized peptide
SMGGMAASGGYWIS

SEQ ID NO: 45 - protein, 11 aa, Artificial
  chemically synthesized peptide
AKGYWVENGEI

SEQ ID NO: 46 - protein, 11 aa, Artificial
  chemically synthesized peptide
TTIQVDGSEKK

SEQ ID NO: 47 - protein, 11 aa, Artificial
  chemically synthesized peptide
SQDGNNHQFTT

SEQ ID NO: 48 - protein, 11 aa, Artificial
  chemically synthesized peptide
DPQWKYSQETA

SEQ ID NO: 49 - protein, 13 aa, Artificial
  chemically synthesized peptide
DNFMKDVLRLIEQ

SEQ ID NO: 50 - protein, 12 aa, Artificial
  chemically synthesized peptide
EQYVSSHTHAMK

SEQ ID NO: 51 - protein, 14 aa, Artificial
  chemically synthesized peptide
SPYGPEARAELSSR

SEQ ID NO: 52 - protein, 12 aa, Artificial
  chemically synthesized peptide
GNMDDYDWMNEN

SEQ ID NO: 53 - protein, 12 aa, Artificial
  chemically synthesized peptide
WVRAHDNDEHYM

SEQ ID NO: 54 - protein, 14 aa, Artificial
  chemically synthesized peptide
FNLADVAICIGAAL

SEQ ID NO: 55 - protein, 14 aa, Artificial
  chemically synthesized peptide
SRHHWGSDLDIYDP

SEQ ID NO: 56 - protein, 15 aa, Artificial
  chemically synthesized peptide
WGCLHEIAHGYQGGF

SEQ ID NO: 57 - protein, 13 aa, Artificial
  chemically synthesized peptide
LILFEPANFNSMG

SEQ ID NO: 58 - protein, 10 aa, Artificial
  chemically synthesized peptide
GYGDFKLLAA

SEQ ID NO: 59 - protein, 11 aa, Artificial
  chemically synthesized peptide
KPQFTSYSVVD

SEQ ID NO: 60 - protein, 10 aa, Artificial
  chemically synthesized peptide
DELNSIVENN

SEQ ID NO: 61 - protein, 10 aa, Artificial
  chemically synthesized peptide
TSMVHEPNFD

SEQ ID NO: 62 - protein, 10 aa, Artificial
  chemically synthesized peptide
EGHLLSPLLD

SEQ ID NO: 63 - protein, 10 aa, Artificial
  chemically synthesized peptide
TAYHEAGHAI

SEQ ID NO: 64 - protein, 10 aa, Artificial
  chemically synthesized peptide
STGGSKMYVK

SEQ ID NO: 65 - protein, 10 aa, Artificial
  chemically synthesized peptide
NRLMTPASTN

SEQ ID NO: 66 - protein, 10 aa, Artificial
  chemically synthesized peptide
ETIVKDTDRD

SEQ ID NO: 67 - protein, 10 aa, Artificial
  chemically synthesized peptide
YWFGKDALEL

SEQ ID NO: 68 - protein, 10 aa, Artificial
  chemically synthesized peptide
EKLQTTHFSI

SEQ ID NO: 69 - protein, 10 aa, Artificial
  chemically synthesized peptide
IHLSVQANAV

SEQ ID NO: 70 - protein, 10 aa, Artificial
  chemically synthesized peptide
GKFSLDVDFT

SEQ ID NO: 71 - protein, 11 aa, Artificial
  chemically synthesized peptide
THFGEHPSLKI

SEQ ID NO: 72 - protein, 10 aa, Artificial
  chemically synthesized peptide
DAMSTNKFGV

SEQ ID NO: 73 - protein, 11 aa, Artificial
  chemically synthesized peptide
VGSQNSSNSNR

SEQ ID NO: 74 - protein, 12 aa, Artificial
  chemically synthesized peptide
SSVSPGQGKDSP

SEQ ID NO: 75 - protein, 13 aa, Artificial
  chemically synthesized peptide
GSNISHKKVSYED

SEQ ID NO: 76 - protein, 11 aa, Artificial
  chemically synthesized peptide
LYDLQRSAMVY

SEQ ID NO: 77 - protein, 11 aa, Artificial
  chemically synthesized peptide
YRDKLVGKTVK

SEQ ID NO: 78 - protein, 10 aa, Artificial
  chemically synthesized peptide
PSTADLLSNY

SEQ ID NO: 79 - protein, 10 aa, Artificial
  chemically synthesized peptide
LKWRCPFPDQ

SEQ ID NO: 80 - protein, 8 aa, Artificial
  chemically synthesized peptide
LSYSREQT

SEQ ID NO: 81 - protein, 14 aa, Artificial
  chemically synthesized peptide
LGIAHLLEHLLISF

SEQ ID NO: 82 - protein, 14 aa, Artificial
  chemically synthesized peptide
IRFHIKELENEYYF

SEQ ID NO: 83 - protein, 14 aa, Artificial
  chemically synthesized peptide
MFGFCYLSHWKCVI

SEQ ID NO: 84 - protein, 14 aa, Artificial
  chemically synthesized peptide
CLVSFYDSGGNIPT

SEQ ID NO: 85 - protein, 15 aa, Artificial
  chemically synthesized peptide
VEVNQLLESECGMFI

SEQ ID NO: 86 - protein, 12 aa, Artificial
  chemically synthesized peptide
LAHELIHAGHRL

SEQ ID NO: 87 - protein, 11 aa, Artificial
  chemically synthesized peptide
FEELRTFGGHD

SEQ ID NO: 88 - protein, 12 aa, Artificial
  chemically synthesized peptide
YNQYTEEEKNNI

SEQ ID NO: 89 - protein, 9 aa, Artificial
  chemically synthesized peptide
HQFNNIAKL

SEQ ID NO: 90 - protein, 9 aa, Artificial
  chemically synthesized peptide
VASNWYNRQ

SEQ ID NO: 91 - protein, 9 aa, Artificial
  chemically synthesized peptide
IERSSRTLG

SEQ ID NO: 92 - protein, 12 aa, Artificial
  chemically synthesized peptide
LMHELIHVLHGL

SEQ ID NO: 93 - protein, 11 aa, Artificial
  chemically synthesized peptide
AEELYTFGGQD

SEQ ID NO: 94 - protein, 12 aa, Artificial
  chemically synthesized peptide
YNIYSEKEKSNI

SEQ ID NO: 95 - protein, 13 aa, Artificial
  chemically synthesized peptide
HRFYESGIVFEEY

SEQ ID NO: 96 - protein, 12 aa, Artificial
  chemically synthesized peptide
KDYFCISKWYLK

SEQ ID NO: 97 - protein, 12 aa, Artificial
  chemically synthesized peptide
EVKRKPYNLKLG

SEQ ID NO: 98 - protein, 12 aa, Artificial
  chemically synthesized peptide
LMHELNHTMHNL

SEQ ID NO: 99 - protein, 11 aa, Artificial
  chemically synthesized peptide
YAEIYAFGGPT

SEQ ID NO: 100 - protein, 12 aa, Artificial
  chemically synthesized peptide
YKKYSGSDKENI

SEQ ID NO: 101 - protein, 12 aa, Artificial
  chemically synthesized peptide
LMHELTHSLHQL

SEQ ID NO: 102 - protein, 11 aa, Artificial
  chemically synthesized peptide
FEELYTFGGLD

SEQ ID NO: 103 - protein, 12 aa, Artificial
  chemically synthesized peptide
YKKYSGSDKENI

SEQ ID NO: 104 - protein, 12 aa, Artificial
  chemically synthesized peptide
LMHELIHSLHGL

SEQ ID NO: 105 - protein, 11 aa, Artificial
  chemically synthesized peptide
IEEFLTFGGTD

SEQ ID NO: 106 - protein, 12 aa, Artificial
  chemically synthesized peptide
YNSYTLEEKNEL

SEQ ID NO: 107 - protein, 11 aa, Artificial
  chemically synthesized peptide
STWYYTHMRDH

SEQ ID NO: 108 - protein, 12 aa, Artificial
  chemically synthesized peptide
LAHELIHALHGL

SEQ ID NO: 109 - protein, 11 aa, Artificial
  chemically synthesized peptide
LEEFLTFGGQD

SEQ ID NO: 110 - protein, 12 aa, Artificial
  chemically synthesized peptide
YNNYTSDEKNRL

SEQ ID NO: 111 - protein, 11 aa, Artificial
  chemically synthesized peptide
HSNNLVASSWY

SEQ ID NO: 112 - protein, 12 aa, Artificial
  chemically synthesized peptide
YNNIRRNTSSNG

SEQ ID NO: 113 - protein, 12 aa, Artificial
  chemically synthesized peptide
LMHELIHVLHGL

SEQ ID NO: 114 - protein, 11 aa, Artificial
  chemically synthesized peptide
AEELYTFGGHD

SEQ ID NO: 115 - protein, 12 aa, Artificial
  chemically synthesized peptide
YNRYSEEDKMNI

SEQ ID NO: 116 - protein, 9 aa, Artificial
  chemically synthesized peptide
SQWYLRRIS

SEQ ID NO: 117 - protein, 9 aa, Artificial
  chemically synthesized peptide
ENINKLRLG

SEQ ID NO: 118 - protein, 9 aa, Artificial
  chemically synthesized peptide
WGKEEEKRW

SEQ ID NO: 119 - protein, 11 aa, Artificial
  chemically synthesized peptide
PIPETLIAYRR

SEQ ID NO: 120 - protein, 12 aa, Artificial
  chemically synthesized peptide
DTDRDGIPDEWE

SEQ ID NO: 121 - protein, 8 aa, Artificial
  chemically synthesized peptide
GQIDPSVS

SEQ ID NO: 122 - protein, 10 aa, Artificial
  chemically synthesized peptide
DRLWHGFVVD

SEQ ID NO: 123 - protein, 10 aa, Artificial
  chemically synthesized peptide
WHFATFNLAD

SEQ ID NO: 124 - protein, 10 aa, Artificial
  chemically synthesized peptide
GYGDVKFLAA

SEQ ID NO: 125 - protein, 10 aa, Artificial
  chemically synthesized peptide
SMVDKDWNNS

SEQ ID NO: 126 - protein, 10 aa, Artificial
  chemically synthesized peptide
SDTDKHYQTE

SEQ ID NO: 127 - protein, 10 aa, Artificial
  chemically synthesized peptide
NNLHTGESIK

SEQ ID NO: 128 - protein, 10 aa, Artificial
  chemically synthesized peptide
HEKNVMNDAW

SEQ ID NO: 129 - protein, 10 aa, Artificial
  chemically synthesized peptide
HTDVVPPGDA

SEQ ID NO: 130 - protein, 10 aa, Artificial
  chemically synthesized peptide
EEASAHNGTV

SEQ ID NO: 131 - protein, 10 aa, Artificial
  chemically synthesized peptide
NATIHKINEC

SEQ ID NO: 132 - protein, 9 aa, Artificial
  chemically synthesized peptide
YNYHGKHEF

SEQ ID NO: 133 - protein, 10 aa, Artificial
  chemically synthesized peptide
HVDTSPDCSG

SEQ ID NO: 134 - protein, 10 aa, Artificial
  chemically synthesized peptide
EEVGKGAKHF

SEQ ID NO: 135 - protein, 10 aa, Artificial
  chemically synthesized peptide
DGGGVGELEF

SEQ ID NO: 136 - protein, 11 aa, Artificial
  chemically synthesized peptide
TMTEEAGMDGA

SEQ ID NO: 137 - protein, 10 aa, Artificial
  chemically synthesized peptide
GPTITGPHSP

SEQ ID NO: 138 - protein, 11 aa, Artificial
  chemically synthesized peptide
GGRFNGGHASH

SEQ ID NO: 139 - protein, 6 aa, Artificial
  chemically synthesized peptide
HFLEHM

SEQ ID NO: 140 - protein, 6 aa, Artificial
  chemically synthesized peptide
HYLEHM

SEQ ID NO: 141 - protein, 9 aa, Artificial
  chemically synthesized peptide
TTLGADNGI

SEQ ID NO: 142 - protein, 8 aa, Artificial
  chemically synthesized peptide
GLKGGHSG

SEQ ID NO: 143 - protein, 6 aa, Artificial
  chemically synthesized peptide
NAIPRE

SEQ ID NO: 144 - protein, 7 aa, Artificial
  chemically synthesized peptide
GQKVKQG

SEQ ID NO: 145 - protein, 7 aa, Artificial
  chemically synthesized peptide
GQKVKRG

SEQ ID NO: 146 - protein, 13 aa, Artificial
  chemically synthesized peptide
IHECGHAVYEQNI

SEQ ID NO: 147 - protein, 13 aa, Artificial
  chemically synthesized peptide
VHETGHARYEQNL

SEQ ID NO: 148 - protein, 7 aa, Artificial
  chemically synthesized peptide
IHESQSL

SEQ ID NO: 149 - protein, 6 aa, Artificial
  chemically synthesized peptide
RITTRY

SEQ ID NO: 150 - protein, 9 aa, Artificial
  chemically synthesized peptide
LHEIAHGYQ

SEQ ID NO: 151 - protein, 7 aa, Artificial
  chemically synthesized peptide
GNSGGAL

SEQ ID NO: 152 - protein, 8 aa, Artificial
  chemically synthesized peptide
EGIGLAIP

SEQ ID NO: 153 - protein, 8 aa, Artificial
  chemically synthesized peptide
IGIGFAIP

SEQ ID NO: 154 - protein, 8 aa, Artificial
  chemically synthesized peptide
PASMTKIM

SEQ ID NO: 155 - protein, 8 aa, Artificial
  chemically synthesized peptide
PASLTKIM

SEQ ID NO: 156 - protein, 6 aa, Artificial
  chemically synthesized peptide
SGNDAS

SEQ ID NO: 157 - protein, 7 aa, Artificial
  chemically synthesized peptide
MRVISVV

SEQ ID NO: 158 - protein, 7 aa, Artificial
  chemically synthesized peptide
MRLISVV

SEQ ID NO: 159 - protein, 6 aa, Artificial
  chemically synthesized peptide
SMMPTL

SEQ ID NO: 160 - protein, 12 aa, Artificial
  chemically synthesized peptide
DYVKRIIGLPGD

SEQ ID NO: 161 - protein, 12 aa, Artificial
  chemically synthesized peptide
DYIKRVVGLPGD

SEQ ID NO: 162 - protein, 7 aa, Artificial
  chemically synthesized peptide
FVHGGPG

SEQ ID NO: 163 - protein, 7 aa, Artificial
  chemically synthesized peptide
FIHGGPG

SEQ ID NO: 164 - protein, 6 aa, Artificial
  chemically synthesized peptide
GFFLNN

SEQ ID NO: 165 - protein, 8 aa, Artificial
  chemically synthesized peptide
GSPGGNRI

SEQ ID NO: 166 - protein, 8 aa, Artificial
  chemically synthesized peptide
GSPGGSRI

SEQ ID NO: 167 - protein, 6 aa, Artificial
  chemically synthesized peptide
AYSGRC

SEQ ID NO: 168 - protein, 7 aa, Artificial
  chemically synthesized peptide
SLKVEGR

SEQ ID NO: 169 - protein, 7 aa, Artificial
  chemically synthesized peptide
SLKIEGR

SEQ ID NO: 170 - protein, 11 aa, Artificial
  chemically synthesized peptide
LKFSDGSPLTA

SEQ ID NO: 171 - protein, 7 aa, Artificial
  chemically synthesized peptide
KAGLDPE

SEQ ID NO: 172 - protein, 10 aa, Artificial
  chemically synthesized peptide
AHNDEMALGA

SEQ ID NO: 173 - protein, 10 aa, Artificial
  chemically synthesized peptide
AHNDDMAIGA

SEQ ID NO: 174 - protein, 7 aa, Artificial
  chemically synthesized peptide
LKPDLII

SEQ ID NO: 175 - protein, 7 aa, Artificial
  peptide
LKPDVII

SEQ ID NO: 176 - protein, 11 aa, Artificial
  chemically synthesized peptide
QIEQGAPADLF

SEQ ID NO: 177 - protein, 13 aa, Artificial
  chemically synthesized peptide
DVIGHELTHAVTE

SEQ ID NO: 178 - protein, 11 aa, Artificial
  chemically synthesized peptide
EYDTQYSGHGE

SEQ ID NO: 179 - protein, 12 aa, Artificial
  chemically synthesized peptide
ELFRHEFTHYLQ

SEQ ID NO: 180 - protein, 11 aa, Artificial
  chemically synthesized peptide
SAIPGTSEHQT

SEQ ID NO: 181 - protein, 12 aa, Artificial
  chemically synthesized peptide
FGTIHECGHAVY

SEQ ID NO: 182 - protein, 12 aa, Artificial
  chemically synthesized peptide
EGFIHEFGHAVD

SEQ ID NO: 183 - protein, 13 aa, Artificial
  chemically synthesized peptide
EEVGLRGAKTSAN

SEQ ID NO: 184 - protein, 12 aa, Artificial
  chemically synthesized peptide
GTLHEIAHGYQA

SEQ ID NO: 185 - protein, 16 aa, Artificial
  chemically synthesized peptide
HEFTHYLQGRYEVPGL

SEQ ID NO: 186 - protein, 13 aa, Artificial
  chemically synthesized peptide
DVIGHELTHAVTE

SEQ ID NO: 187 - protein, 12 aa, Artificial
  chemically synthesized peptide
ADYTRGQGIETY